(19) United States
(12) Patent Application Publication
(10) Pub. No.: US 2024/0216851 A1
(43) Pub. Date: Jul. 4, 2024

(54) PROCESS OF TREATING A FEED IN AN ADSORPTION SEPARATION ZONE COMPRISING A TRIM BED

(71) Applicant: UOP LLC, Des Plaines, IL (US)

(72) Inventors: Jeffrey L. Pieper, Schaumburg, IL (US); Uday Kiran Erraguntla, Gurugram (IN); Sanjeev M. Rao, Buffalo Grove, IL (US)

(21) Appl. No.: 18/394,962

(22) Filed: Dec. 22, 2023

(30) Foreign Application Priority Data
Dec. 31, 2022 (IN) 202211077658

(51) Int. Cl. B01D 53/04; B01D 3/14; B01J 20/34; C07C 7/12

(52) U.S. Cl. CPC B01D 53/0423; B01D 3/143; B01J 20/3458; C07C 7/12; B01D 2257/70; B01D 2259/40086; B01D 2259/402

(57) ABSTRACT

A process of treating a feed in an adsorption separation zone comprising at least two adsorbent beds and a trim bed is disclosed. The process comprises displacing feed from a first adsorbent bed to a trim bed by feeding a desorbent from a second adsorbent bed to the first adsorbent bed while displacing spent desorbent from the trim bed. After discontinuing the first adsorbent bed displacement, an adsorber trim fluid to the first adsorbent bed to recover the remaining treated feed in the head. After discontinuing adsorber trim fluid to the first adsorbent bed; a trim displacement fluid is passed to the trim bed to displace feed from the trim bed.

### FIELD

The field is related to a process of treating a feed in an adsorption separation zone. Particularly, the field relates to a process of treating a feed in an adsorption separation zone comprising a trim bed.

### BACKGROUND

Adsorptive separation is applied to the recovery of a variety of hydrocarbon and other chemical products. Chemical separations using this approach include: the separation of mixtures of aromatics into specific aromatic isomers, the separation of linear from nonlinear aliphatic and olefinic hydrocarbons, the separation of either paraffins or aromatics from a feed mixture comprising both aromatics and paraffins, the separation of chiral compounds for use in pharmaceuticals and fine chemicals, the separation of oxygenates such as alcohols and ethers, and the separation of carbohydrates such as sugars.

Packed beds of adsorbent materials are typically used in adsorption processes. Adsorbent materials are generally in the form of spherical beads, or pellets. Adsorbent materials are typically oxygen-containing compounds, carbon-containing compounds, or polymer-based compounds. Oxygen-containing compounds can be, for example, hydrophilic and polar, including materials such as silica gel and zeolites. Carbon-based compounds can be, for example, hydrophobic and non-polar, including materials such as activated carbon and graphite. Polymer-based compounds can be, for example, polar or non-polar functional groups in a porous polymer matrix.

In operation, a process stream is introduced into an adsorbent bed, and the adsorbent material contained therein removes a desired or undesired component from the stream as it filters through the adsorbent bed. After a given time period, the adsorbent material becomes saturated with the sorbate, and the adsorption process in that adsorbent bed must be stopped in order to remove the sorbate and regenerate the adsorbent. After a regeneration cycle is complete, a new adsorption cycle can begin. Adsorption processes often operate in a swing bed arrangement so that one or more beds are active, and one or more beds are being regenerated.

Several problems can occur during operation with a swing bed arrangement. First, feed can be lost when switching a bed from active to regeneration. In addition, there is a risk that when the adsorbent bed is full, some of the material which is supposed to be adsorbed in the adsorbent bed will flow through the adsorbent bed and not be captured.

Thus, there exists a need for an improved process for operating an adsorption separation zone.

### SUMMARY

The present disclosure provides a process and an apparatus for treating a feed in an adsorption separation zone comprising at least two adsorbent beds and a trim bed. Generally, a two-bed unit may be used in a linear alkylbenzene (LAB) complex. It was noticed that the operation of the two-bed unit has resulted in increased feed loss and breakthrough of heavy aromatics at the bottom of the trim adsorber. Also, the recovery of the treated feed from the adsorbent beds was also affected in that the feed from the top of the adsorbent beds was not fully recovered and residual feed remaining at the top and/or other part of the adsorbent beds. The present process comprises displacing feed from a first adsorbent bed to a trim bed by feeding a desorbent from a second adsorbent bed to the first adsorbent bed while displacing spent desorbent from the trim bed. After discontinuing the first adsorbent bed displacement a trim fluid is passed to the first adsorbent bed to recover the remaining feed material in the top head, later the trim fluid is passed to the trim bed resulting in greater feed recovery and consequent adsorption of unwanted materials therefrom.

### BRIEF DESCRIPTION OF THE DRAWINGS

The various embodiments will hereinafter be described in conjunction with the following FIGURES, wherein like numerals denote like elements.

FIGS. 1-5 are schematic diagrams of a process of treating a feed in an adsorption separation zone comprising at least two adsorbent beds and a trim bed in accordance with an exemplary embodiment.

FIGS. 6-10 are schematic diagrams of the process of FIG. 1-4 with an additional embodiment of a trim displacement system.

FIG. 11 is a schematic diagram of a process of treating a feed in an adsorption separation zone comprising at least two adsorbent beds and a trim bed in accordance with yet another exemplary embodiment including a heavy aromatics column.

### DETAILED DESCRIPTION

Typically, a LAB complex contains an adsorptive separation process to adsorb heavy aromatics and prevent the breakthrough of heavy aromatics to the downstream units. In a typical scheme nowadays, the LAB complex includes a six-bed unit (older designs) or a two-bed unit with a trim bed (newer designs). The LAB complex with two-bed unit contains two adsorbers operating alternately with an additional trim adsorber vessel to capture heavy aromatics that have desorbed from the main adsorber during displacement and trimming with benzene. Applicants discovered that the operation of the two-bed unit has resulted in increased feed loss and breakthrough of heavy aromatics at the bottom of the trim adsorber. Applicants have found that the breakthrough of heavy aromatics is attributed to the use of benzene to trim the smaller adsorber in order to recover the valuable feed components. Benzene being a strong desorbent also desorbs adsorbed heavy aromatics. In accordance with the present disclosure, a process of treating a feed in an adsorption separation zone comprising at least two adsorbent beds and a trim bed is disclosed to avoid the desorption of heavy aromatics in the feed displacement and trim step.

In the two-bed unit, along with the two main adsorbent beds, a trim bed is employed. The trim bed will catch the small amounts of heavy aromatics remaining in the adsorbent bed during the switch over from the spent adsorbent bed to the fresh adsorbent bed. In addition, any adsorbed heavy aromatics that desorb from the spent adsorbent bed during the displacement step would be adsorbed onto the fresh adsorbent bed. The trim bed is regenerated together with the spent adsorber. The process also provides an improved recovery of feed material from the main adsorbent beds wherein any residual feed which remain in the head and connected piping of main adsorber will also be flushed out to the trim adsorber and recovered.

When regeneration of an adsorbent bed is initiated, the untreated feed will switch from a spent adsorbent bed to the fresh adsorbent bed. The flow path is altered so that the desorbent displaced from the fresh adsorbent bed is sent to the spent adsorbent bed. Treated feed displaced from the spent adsorbent bed is sent through the trim bed before being sent on to a downstream process. The trim bed will re-adsorb any desorbed material, such as heavy aromatics, from the spent adsorbent bed during the displacement. After substantially all of the desorbent has been displaced from the fresh adsorbent bed and the volume has been filled with feed, the flow path is changed so that treated feed from the fresh adsorbent bed is sent directly to the downstream process. By “substantially all,” we mean at least about 90 vol %, or at least about 93 vol %, or at least about 95 vol %, or at least about 97 vol %, or at least about 98 vol %.

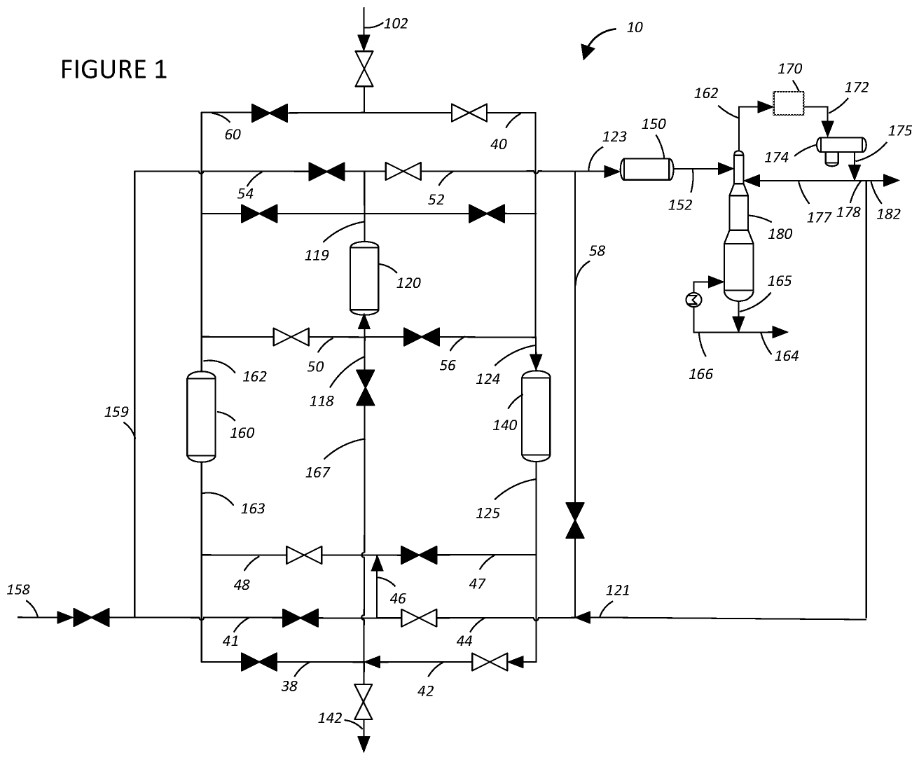

In FIG. 1, in accordance with an exemplary embodiment, a process of treating a feed in an adsorption separation unit 10 comprising at least two adsorbent beds 140, 160 and a trim bed 120 is shown. In FIG. 1, the adsorption separation unit 10 comprises at least two adsorbent beds and a trim bed in selective communication. In accordance with the exemplary embodiment, the adsorption separation unit 10 comprises a first adsorbent bed 140, a second adsorbent bed 160, and a trim bed 120. Untreated feed in line 102 is passed to the top of the first adsorbent bed 140 to remove one or more compounds from the feed to produce treated feed. For example, in a LAB process, a portion of the effluent from the selective hydrogenation process contains heavy aromatics which must be removed. The treated feed exits from the bottom of the first adsorbent bed 140 and passes out through the product outlet line 142. In accordance with an exemplary embodiment of the present disclosure, the treated feed in the product outlet line 142 is passed to a downstream alkylation unit. In FIG. 1, the first adsorbent bed 140 is isolated from the second adsorbent bed 160 and the trim bed 120 because the second adsorbent bed was taking off line after becoming saturated with adsorbed aromatics.

The untreated feed in a feed inlet line 102 is passed through the first adsorbent bed 140 through a first upper adsorbent manifold 124 to produce treated feed. The untreated feed for an LAB process typically comprises about 80 to about 90 wt % paraffins, about 1, preferably about 10 wt %, to about 15 wt % olefins, and typically about 0.5 wt % to about 5 wt % aromatics. The treated feed passes out through a product outlet line 142. A desorbent in line 121 is passed through the second adsorbent bed 160 and the trim bed 120 to remove adsorbed material from the second adsorbent bed and the trim bed to regenerate the second adsorbent bed and the trim bed producing spent desorbent. The spent desorbent passes out through a spent desorbent outlet line 123.

The first adsorbent bed 140 is in downstream communication with a feed inlet line 102, through a first adsorbent inlet line 40, and the first upper adsorbent manifold 124. The product outlet line 142 is in downstream communication with the first adsorbent bed 140 through the first adsorbent outlet line 42 and the lower adsorbent manifold 125. The second adsorbent bed 160 is in downstream communication with the desorbent line 121 through a first transfer line 44, a tie line 46, a second transport line 48, and a second lower adsorbent manifold 163. The trim bed 120 is in downstream communication with the second adsorbent bed 160 through a second upper adsorbent manifold 162, a second desorbent push line 50 and a lower trim manifold 118. Desorbent exits the trim bed 120 through an upper trim manifold 119, a first desorbent exit line 52 and a spent desorbent outlet line 123.

In accordance with the present disclosure, the spent desorbent stream in line 123 is typically further treated to provide the desorbent stream. In accordance with an exemplary embodiment shown in FIG. 1, the spent desorbent stream in line 123 may be passed through an open valve thereon to a mixing drum 150. From the mixing drum 150, a spent desorbent stream in line 152 is passed to a desorbent column 180. In the desorbent column 180, the spent desorbent stream is separated to provide an overhead stream comprising the desorbent in line 162 and a bottoms stream comprising aromatics in line 165. The overhead stream comprising the desorbent in line 162 is first cooled via a cooler 170 and a condensed overhead stream is passed to an overhead receiver 174. An overhead desorbent stream in line 175 is withdrawn from the receiver 174, separated into a reflux stream 177 and a condensed desorbent stream in line 178. A desorbent stream can be taken in line 121 to the process while a desorbent product stream may be taken in line 182. The reflux stream 177 is passed to the desorbent column 180. The bottoms stream comprising aromatics in line 165 is separated into a reboiling stream in line 166 and an aromatics stream in line 164 which is withdrawn. The heavy paraffins routed to the desorbent column 180 in the spent desorbent stream in line 123 will be recovered in the net bottoms stream 164. If paraffins are in the bottoms stream 164, it will be routed to a hydrotreating unit for saturating any unsaturated components such as olefins and aromatics to meet the feed specifications and then the stream is further processed in a process unit for recovery of normal paraffins from isoparaffins. That way paraffins in the spent desorbent stream 123 will not be lost. For simplicity, the desorbent column is not shown in subsequent FIGS.

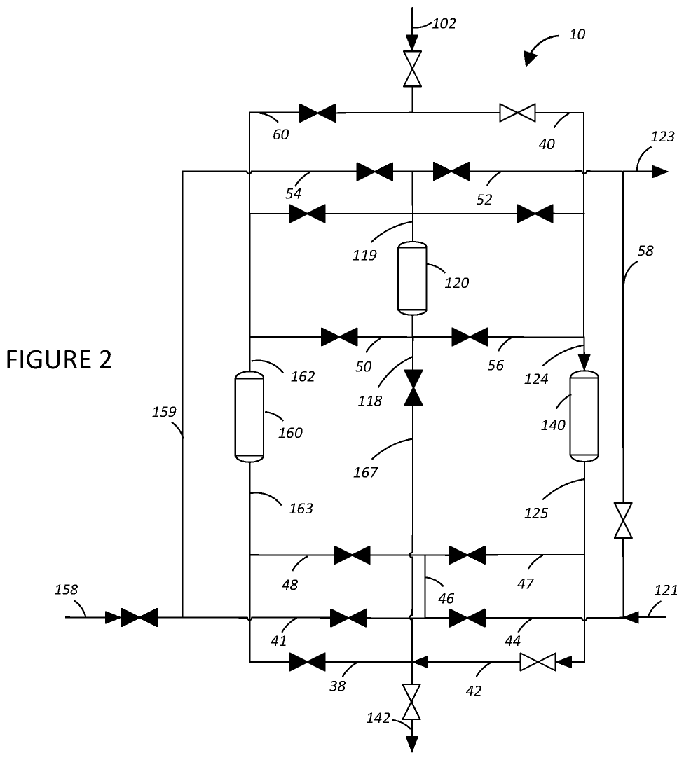

As shown in FIG. 2, when the adsorbed material is desorbed from the second adsorbent bed 160 and the trim bed 120, the flow of the desorbent to the second adsorbent bed and the trim bed is stopped and the second adsorbent bed and the trim bed are isolated by shutting valves on the first transfer line 44, the second transport line 48, the second desorbent push line 50 and the first desorbent exit line 52 as shown in FIG. 2. A valve on the desorbent bypass line 58 is opened so desorbent bypasses to the downstream process in the spent desorbent exit line 123. The untreated feed continues to pass through the first adsorbent bed 140 and the treated feed continues to pass out through the product outlet line 142.

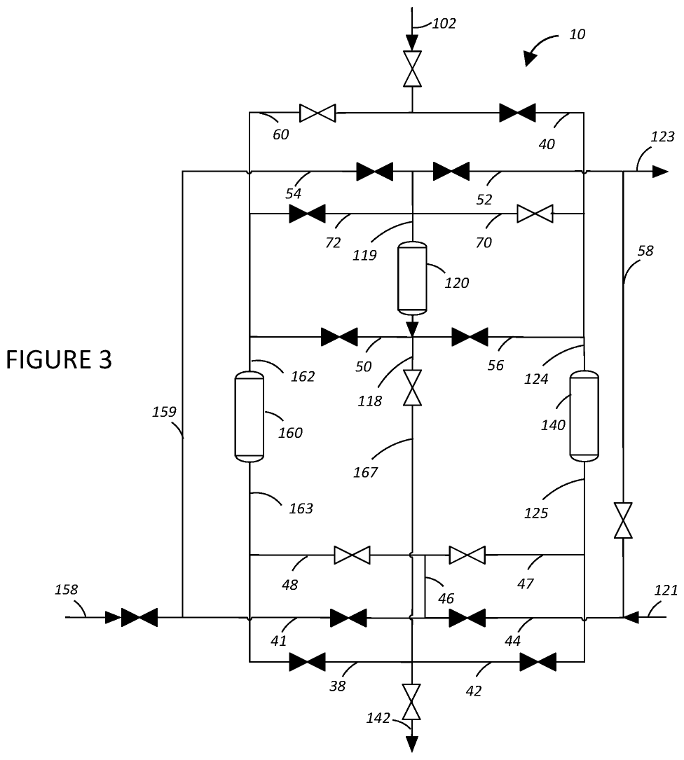

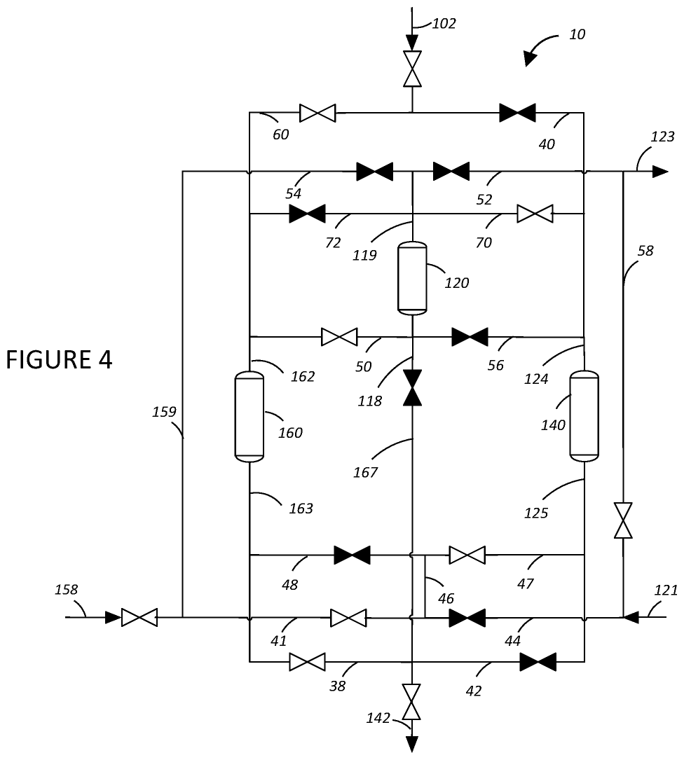

Turning to FIG. 3, when the adsorbent in the first adsorbent bed 140 is fully saturated, the flow of the untreated feed to the first adsorbent bed 140 is then stopped by closing the valve on the first adsorbent inlet line 40, and the untreated feed is passed through the second adsorbent bed 160 by opening the valve on the second adsorbent inlet line 60 to produce additional treated feed. The first adsorbent bed 140 contains treated feed, so a displacement step for the first adsorbent bed 140 has to be performed to not lose the treated feed. In the displacement step for the first adsorbent bed 140, desorbent from the second adsorbent bed 160 is transferred to the first adsorbent bed 140 to displace the treated feed from the first adsorbent bed 140 to the trim bed 120 while displacing desorbent from the trim bed 120. At this stage, the second adsorbent bed 160 is in on-line mode and flow through the first adsorbent bed 140 is reversed. In this arrangement, the second adsorbent bed 160 is in downstream communication with the feed inlet line 102 via the second adsorbent inlet line 60 and a second upper adsorbent manifold 162. The first adsorbent bed 140 is in downstream communication with the second adsorbent bed 160 via the second lower adsorbent manifold 163, the second transport line 48, the first transport line 47 and the first lower adsorbent manifold 125. The trim bed 120 is in downstream communication with the first adsorbent bed 140 through the first upper manifold 124, the first trim feed line 70 and the upper trim manifold 119. The product outlet line 142 is in downstream communication with the trim bed 120 through the lower trim manifold 118 and the lower trim line 167 with an open valve thereon. The reception of feed into the second adsorbent bed 160 displaces the desorbent in the second adsorbent bed 160 and the desorbent from the second adsorbent bed passes to the first adsorbent bed 140. In turn, the desorbent from the second adsorbent bed 160 displaces the treated feed in the first adsorbent bed 140. The treated feed from the first adsorbent bed 140 passes to the trim bed 120 where the treated feed from the first adsorbent bed 140 displaces the desorbent in the trim bed. The desorbent from the trim bed 120 and the treated feed from the first adsorbent bed 140 pass out through the product outlet line 142. Displacement of the first adsorbent bed 140 is discontinued at a point that assures no untreated feed passes from second adsorbent bed 160 to the first adsorbent bed 140 in which heavy aromatics would not be adsorbed due to the saturation of the adsorbent in the first adsorbent bed. However, some small amount of treated feed may be left in the first adsorbent bed 140 as a result. Accordingly, an adsorber trim fluid from line 158 is used to displace treated feed from the first adsorbent bed 140 as shown in FIG. 4.

In FIG. 4, a valve on the adsorber trim fluid line 158 is opened to enable the adsorber trim fluid to flow through a second transfer line 41 through the tie line 46 through a first transport line 47 up through the first lower adsorption manifold 125 through the first adsorption bed 140 to displace treated feed remaining in the top or head of the adsorber 140. From the first adsorption bed 140, treated feed is displaced through the first upper adsorption manifold 124, through the first trim feed line 70 to the upper trim manifold 119 to the trim bed 120. Treated feed leaves the trim bed 120 through the lower trim manifold 118, through the second desorbent push line 50, and the second upper adsorbent manifold 162 and into the adsorber 160. In an exemplary embodiment, the treated feed from the trim bed 120 in the second push line 50 from the lower trim manifold 118 may be combined with the untreated feed from line 102 in the second upper adsorbent manifold 162 to provide a combined feed to the second adsorbent bed 160. Treated feed leaves the second adsorbent bed 160 along with treated feed from the trim bed 120 through the lower outlet manifold 163, a second adsorbent outlet line 38 and the open valve on the product outlet 142. The desorbent bypass valve 58 remains open.

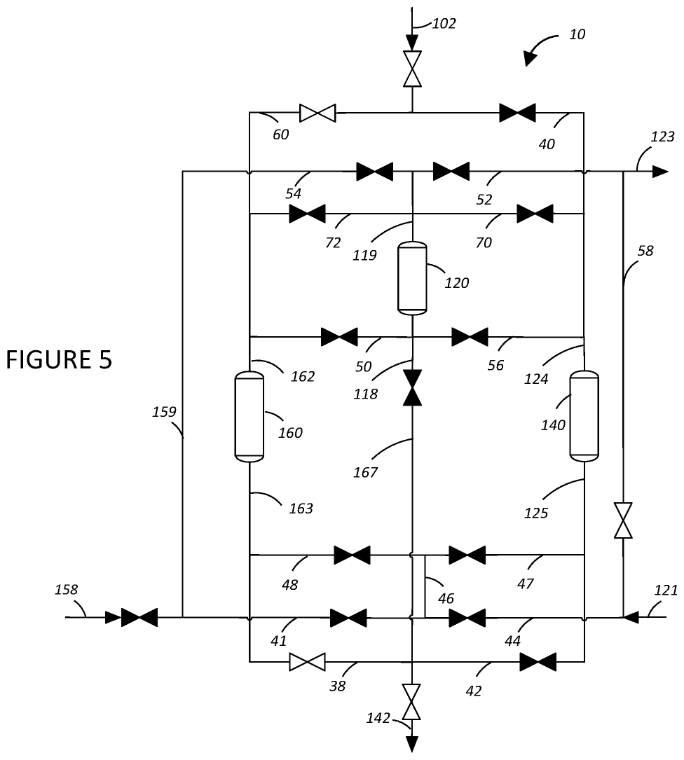

After the first adsorbent bed 140 is prepared for regeneration, FIG. 5 depicts the second adsorption bed 160 on-line adsorbing heavy aromatics from the feed and the first adsorption bed 140 and the trim bed 120 off-line. The second adsorption bed 160 is in downstream communication with the feed inlet line 102 and the product outlet line 142 is in downstream communication with the second adsorption bed. The first adsorption bed 140 and trim bed 120 are isolated from the second adsorption bed 160.

From the displacement of feed in the first adsorbent bed 140 to the trim bed 120, treated feed from the first adsorbent bed 140 employed for pushing the desorbent out of the trim bed is left in the trim bed which should be recovered. Conventionally, benzene desorbent was used to displace treated feed in the trim bed 120. However, we have discovered that the benzene also desorbed heavy aromatics adsorbed in the trim adsorber 120. Desorption of heavy aromatics from the trim adsorber 120 to the second adsorber 160 results in decreased adsorption capacity in the second adsorber 160 which increases regeneration cycles and feed losses. Conversely, reducing the number of regeneration cycles under these circumstances results in breakthrough of heavy aromatics from the bottom of the second adsorber 160 to the product outlet line 142 ruining the purity of the product stream and/or overwhelming downstream separation equipment.

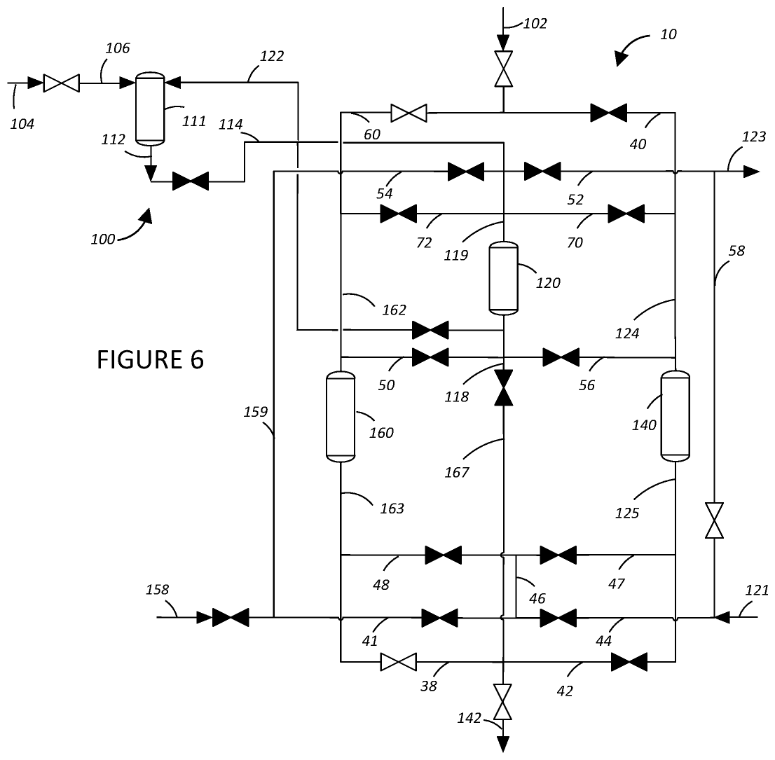

In FIG. 6 an embodiment of a trim displacement system 100 is shown that utilizes a trim displacement fluid that displaces the feed material from the trim adsorber without desorbing heavy aromatics already adsorbed in the adsorbent pores. A trim displacement tank 111 is filled with a trim displacement fluid that may comprise paraffins or heavy aromatics which can displace the treated feed without desorbing heavy aromatics. In the subsequent steps, the trim displacement of the trim bed 120 is performed.

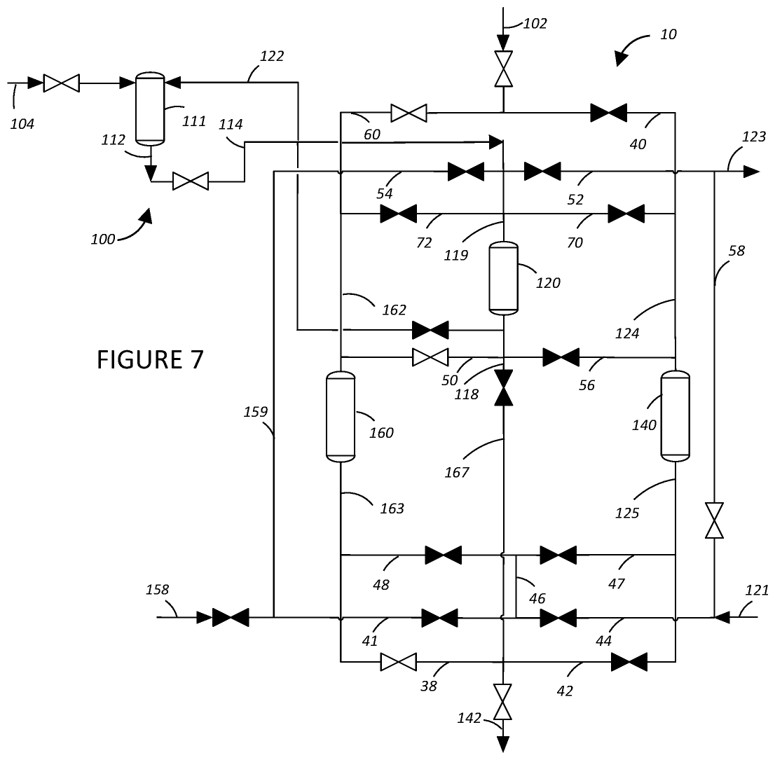

As shown in FIG. 7, a trim displacement fluid in line 114 is passed to the trim bed 120 to perform a trim displacement of feed left in the trim bed 120 that was left there after displacing the feed remaining in the first adsorbent bed 140 to the trim bed. A trim displacement fluid is passed to the trim bed 120 by opening the valve on line 114 to the upper trim manifold 119 which feeds the trim bed 120. In accordance with the present disclosure, the trim displacement fluid may be selected from a heavy aromatics stream or a paraffinic stream. In accordance with an exemplary embodiment, the trim displacement fluid is a heavy aromatics stream taken from a trim displacement fluid drum 111. In accordance with another exemplary embodiment, the heavy aromatics stream is selected from one or more of single-ring aromatics or two-ring aromatics which are mono-alkylated or polyalkylated. A heavy aromatics stream in line 104 may be passed to the trim displacement tank 111. From the trim displacement tank 111, a bottoms stream in line 112 is withdrawn and the trim displacement fluid in line 114 is transported to the upper trim manifold 119.

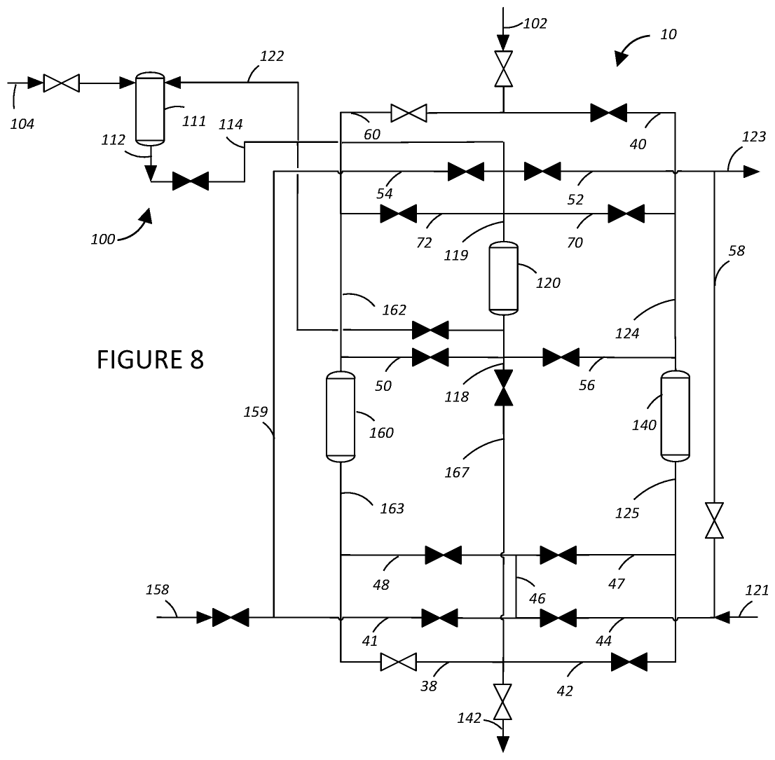

When the trim displacement fluid in line 114 is passed through the trim bed 120, it pushes the treated feed in the trim bed 120 out of it. The trim displacement fluid passes through the trim bed 120 while pushing the feed material out of the trim bed 120 through bottom of the trim bed 120. The treated feed from the bottom of the trim bed 120 in the lower trim manifold 118 is then passed through the second push line 50 and the second upper adsorbent manifold 162 to the second adsorbent bed 160. In an exemplary embodiment, the treated feed from the trim bed 120 in the second push line 50 from the lower trim manifold 118 may be combined with the untreated feed from line 102 in the second upper adsorbent manifold 162 to provide a combined feed to the second adsorbent bed 160. The combined feed in the upper adsorbent manifold 162 is passed to the second adsorbent bed 160 in which the untreated feed is treated to provide the treated feed which may be withdrawn in the product line 142 along with treated feed from the trim bed 120. When the trim displacement fluid in line 114 completely displaces the feed from the trim bed 120, the trim displacement of the trim bed 120 with the first trim displacement fluid in line 114 is discontinued as shown in FIG. 8 by closing the valve for the trim fluid on line 114.

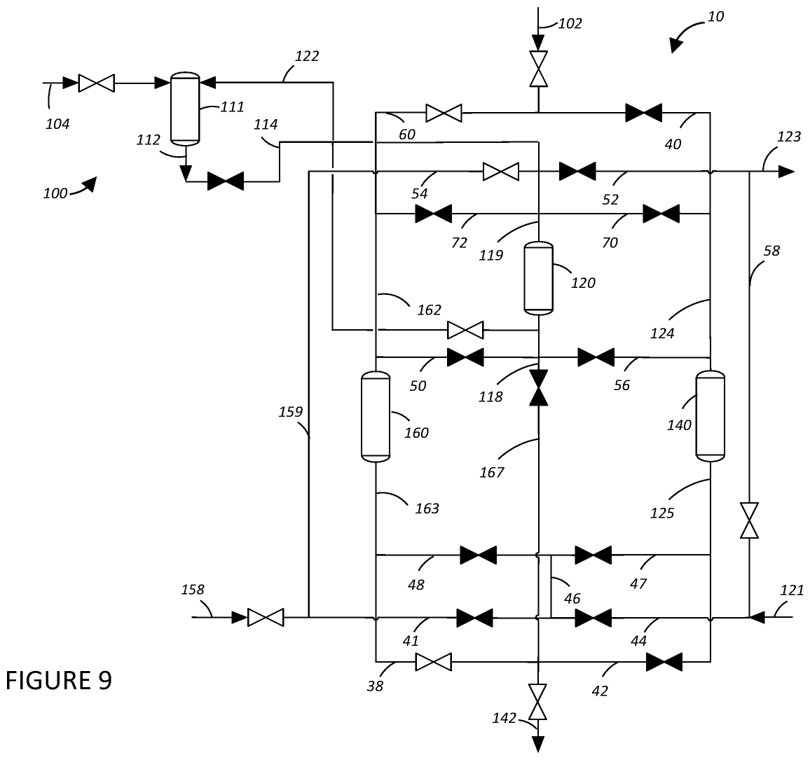

If a heavy aromatics stream is used as the trim displacement fluid, after the trim displacement of the trim bed 120 with the trim displacement fluid in line 114 is completed, a second trim displacement of the trim bed 120 with the adsorber trim fluid is performed. If a paraffin stream is used as the trim displacement fluid, the second trim displacement step with adsorber trim fluid is not needed for the trim adsorber 120 because paraffins can be processed downstream of line 123 in a desorbent column described hereinafter. As shown in FIG. 9, the adsorber trim fluid in the adsorber trim fluid line 158 is passed through an open valve thereon to the trim bed 120 via line 159 through adsorber trim fluid inlet line 54 with an open valve thereon and through the upper trim manifold 119. The adsorber trim fluid performs a trim displacement of the trim displacement fluid from the trim bed 120 without desorbing heavy aromatics. In accordance with the present disclosure, the adsorber trim fluid is a different fluid than the trim displacement fluid. When the displacement of the trim bed 120 with the adsorber trim fluid is performed, a spent trim displacement fluid is displaced from the trim bed 120 into the lower trim manifold 118 as the adsorber trim fluid moves through the trim bed 120. For this, the valve on a return line 122 is opened to release the spent trim fluid in the lower trim manifold 118 from the trim bed. The spent trim fluid in the return line 122 is passed to the trim displacement fluid tank 111.

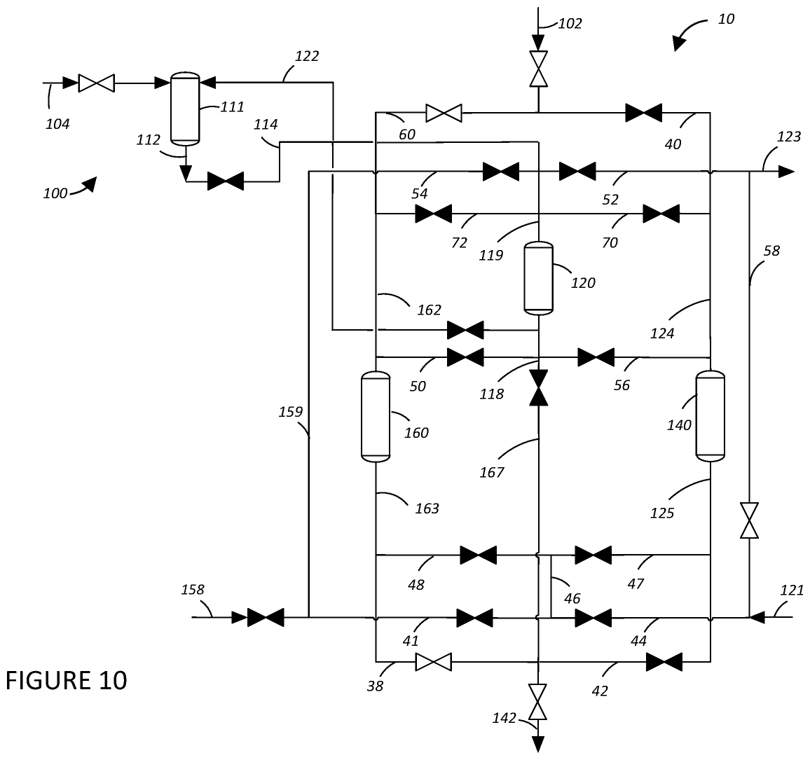

When the spent adsorber trim fluid from the adsorber trim fluid line 158 completely removes the spent trim fluid from the trim bed 120, the trim displacement of the trim bed 120 by the adsorber trim fluid in line 158 is discontinued as shown in FIG. 10 by closing the valve on the adsorber trim fluid inlet line 54 and perhaps the valve on the adsorber trim fluid line 158. The valve on the return line 122 may also be closed.

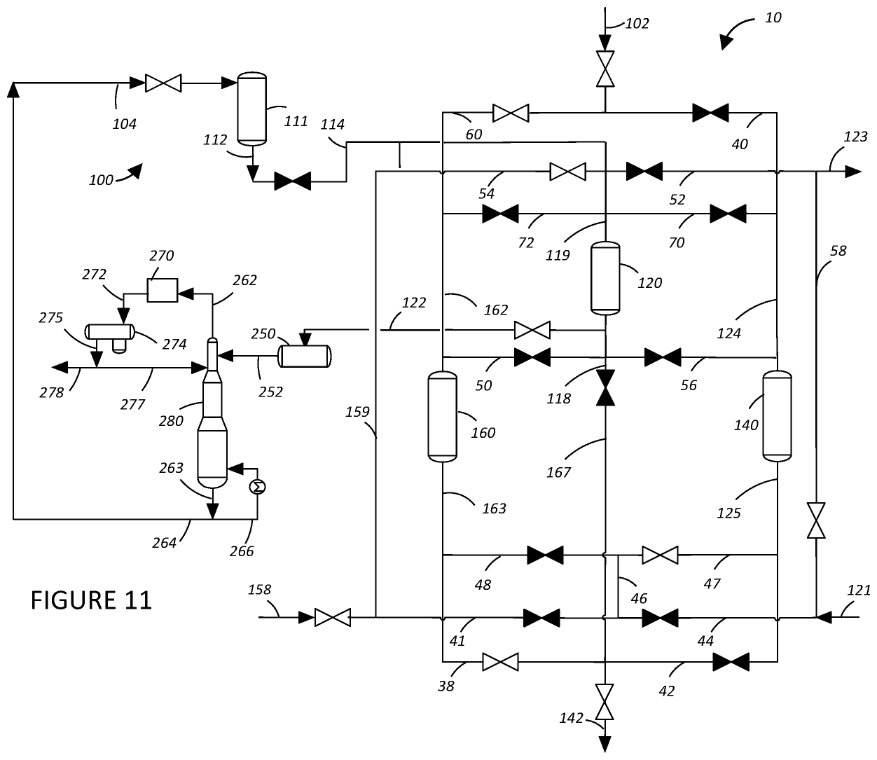

In the embodiment wherein the trim displacement fluid is a heavy aromatics stream, the spent trim displacement fluid from the trim bed 120 may be treated to recover the heavy aromatics from the spent trim displacement fluid. In an exemplary embodiment shown in FIG. 11, the spent trim displacement fluid from the trim bed 120 is passed in the return line 122 to a spent trim displacement fluid tank 250. From the spent trim displacement fluid tank 250, a spent trim displacement fluid in line 252 is passed to the aromatics fractionation column 280. In the aromatics fractionation column 280, the spent trim displacement fluid stream is separated to provide an overhead stream comprising benzene in line 262 and a bottoms stream comprising heavy aromatics in line 263. The overhead stream comprising the benzene in line 262 is cooled via a cooler 270 and a condensed overhead stream is passed to an overhead receiver 274. An overhead benzene stream in line 275 is withdrawn from the receiver 274, separated into a reflux stream 277 and a net benzene stream in line 278 which can be recycled to the desorbent line 121. The reflux stream 277 is passed to the heavy aromatics column 280. The bottoms stream comprising heavy aromatics in line 263 is separated into a reboiling stream in line 266 and a heavy aromatics stream in line 264. The bottoms stream in line 264 comprising heavy aromatics may be recycled to the heavy aromatics surge drum 111 in line 104.

The process continues until the adsorbent in the second adsorbent bed 160 is fully saturated. Then the process recycles as untreated feed is reconnected to the first adsorbent bed 140 with desorption of the second adsorbent bed 160 and the trim bed 120 proceeding.

In accordance with an aspect of the present disclosure, the feed 102 comprises olefins, paraffins, and aromatics. In accordance with an embodiment of the present disclosure, the feed 102 comprises an intermediate feed that may be generated by a paraffin dehydrogenation unit. In accordance with an exemplary embodiment of the present disclosure, the feed 102 comprises from about 80% to about 90% paraffins, from about 10% to about 15% olefins, and from about 0.5% to about 5% aromatics.

In accordance with some embodiments, the desorbent stream is a benzene stream.

In accordance with an exemplary embodiment of the present disclosure, the adsorber trim fluid is benzene which may be obtained from a desorbent or heavy aromatics column overhead.

In accordance with another exemplary embodiment of the present disclosure, the first trim displacement fluid is a paraffinic stream comprising C10 to C24 paraffins.

In an aspect of the present disclosure, the first trim displacement fluid is heavy aromatics. In an exemplary embodiment of the present disclosure, the adsorber trim fluid and the desorbent are benzene. When paraffins are used as first trim fluid, there is no second trim displacement step for the trim adsorber with adsorber trim fluid and the desorption step directly follows displacement of the trim adsorber with the first trim displacement fluid. The contents of the trim bed 120 are routed to a mixing drum 250. When heavy aromatics is used as first trim displacement fluid, the second trim displacement step using adsorber trim fluid will be performed to remove aromatics from the bed and displace them to a spent aromatics tank 150.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect. Further, the figure may include one or more exemplary sensors located on one or more conduits. Nevertheless, there may be sensors present on every stream so that the corresponding parameter(s) can be controlled accordingly.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

### Example

We simulated the disclosed process for a 250 kMTA linear alkylbenzene unit. We calculated that 2 wt % lower n-paraffin feed consumption will be achieved by the disclosed process while producing the same product annually. The improved process will result in $4M savings annually.

### Specific Embodiments

While the following is described in conjunction with specific embodiments, it will be understood that this description is intended to illustrate and not limit the scope of the preceding description and the appended claims.

A first embodiment of the disclosure is a process of treating a feed in an adsorption separation zone comprising at least two adsorbent beds and a trim bed, the process comprising displacing feed from a first adsorbent bed to a trim bed by feeding a desorbent from a second adsorbent bed to the first adsorbent bed while displacing spent desorbent from the trim bed; discontinuing the first adsorbent bed displacement; and passing a trim displacement fluid to the trim bed to displace feed from the trim bed. An embodiment of the disclosure is one, any or all of prior embodiments in this paragraph up through the first embodiment in this paragraph further comprising passing an adsorber trim fluid to the first adsorbent bed to recover the remaining treated feed; and discontinuing adsorber trim fluid to the first adsorbent bed before passing the trim displacement fluid to the trim bed. An embodiment of the disclosure is one, any or all of prior embodiments in this paragraph up through the first embodiment in this paragraph wherein the adsorber trim fluid and/or the desorbent are benzene. An embodiment of the disclosure is one, any or all of prior embodiments in this paragraph up through the first embodiment in this paragraph wherein the trim displacement fluid is selected from a heavy aromatics stream or a paraffinic stream. An embodiment of the disclosure is one, any or all of prior embodiments in this paragraph up through the first embodiment in this paragraph further comprising discontinuing the displacement of feed from the trim bed with the trim displacement fluid; passing an adsorber trim fluid to the trim bed to displace spent trim displacement fluid from the trim bed while withdrawing a spent trim displacement fluid from the trim bed. An embodiment of the disclosure is one, any or all of prior embodiments in this paragraph up through the first embodiment in this paragraph further comprising combining feed displaced from the trim bed with feed that is feeding the second adsorbent bed. An embodiment of the disclosure is one, any or all of prior embodiments in this paragraph up through the first embodiment in this paragraph further comprising passing an adsorber trim fluid to the first adsorbent bed to displace treated feed from the first adsorbent bed to the trim bed. An embodiment of the disclosure is one, any or all of prior embodiments in this paragraph up through the first embodiment in this paragraph wherein the trim displacement fluid comprises heavy aromatics. An embodiment of the disclosure is one, any or all of prior embodiments in this paragraph up through the first embodiment in this paragraph further comprising passing the feed stream through the second adsorbent bed to adsorb material from the feed stream onto the second adsorbent bed. An embodiment of the disclosure is one, any or all of prior embodiments in this paragraph up through the first embodiment in this paragraph further comprising passing a desorbent stream through the first adsorbent bed and the trim bed to remove adsorbed material from the first adsorbent bed and the trim bed to regenerate the first adsorbent bed and the trim bed and withdrawing the spent desorbent stream from the trim bed. An embodiment of the disclosure is one, any or all of prior embodiments in this paragraph up through the first embodiment in this paragraph wherein desorbent is pushed from the second adsorbent bed to the first adsorbent bed by feeding feed to the second adsorbent bed. An embodiment of the disclosure is one, any or all of prior embodiments in this paragraph up through the first embodiment in this paragraph further comprising passing spent trim displacement fluid to an aromatics fractionation column to provide an overhead stream comprising benzene and a bottoms stream comprising heavy aromatics; and recycling the heavy aromatics as a trim fluid. An embodiment of the disclosure is one, any or all of prior embodiments in this paragraph up through the first embodiment in this paragraph further comprising; passing the spent desorbent to a desorbent column to provide a bottoms stream comprising aromatics and an overhead stream comprising the desorbent.

A second embodiment of the disclosure is a process of treating a feed stream in an adsorption separation zone comprising at least two adsorbent beds and a trim bed, the process comprising passing the feed stream through the first adsorbent bed to adsorb material from the feed stream onto the first adsorbent bed; discontinuing passing feed to the first adsorbent bed; passing the feed stream through the second adsorbent bed to adsorb material from the feed stream onto the second adsorbent bed; displacing desorbent in the second adsorbent bed to the first adsorbent bed; displacing feed from the first adsorbent bed to the trim bed; withdrawing a spent desorbent from the trim adsorber; discontinuing displacing feed from the first adsorbent bed; and passing an adsorber trim fluid to the first adsorbent bed to recover remaining feed therein; and passing a trim displacement fluid to the trim bed to displace feed from the trim bed. An embodiment of the disclosure is one, any or all of prior embodiments in this paragraph up through the second embodiment in this paragraph further comprising discontinuing the displacement of feed from the trim bed with the trim displacement fluid; passing an adsorber trim fluid to the trim bed to perform a trim displacement of the trim bed while withdrawing a spent trim displacement fluid from the trim bed; discontinuing the trim displacement of the trim bed with the adsorber trim fluid. An embodiment of the disclosure is one, any or all of prior embodiments in this paragraph up through the second embodiment in this paragraph further comprising combining feed displaced from the trim bed with feed that is feeding the second adsorbent bed. An embodiment of the disclosure is one, any or all of prior embodiments in this paragraph up through the second embodiment in this paragraph further comprising passing a adsorber trim fluid to the trim bed to displace the trim displacement fluid from the trim bed. An embodiment of the disclosure is one, any or all of prior embodiments in this paragraph up through the second embodiment in this paragraph further comprising passing a desorbent stream through the first adsorbent bed and the trim bed to remove adsorbed material from the first adsorbent bed and the trim bed to regenerate the first adsorbent bed and the trim bed and withdrawing the spent desorbent stream from the trim bed.

A third embodiment of the disclosure is a process of treating a feed in an adsorption separation zone comprising at least two adsorbent beds and a trim bed, the process comprising displacing feed from a first adsorbent bed to a trim bed by feeding feed to a second adsorbent bed to displace desorbent from the second adsorbent bed to the first adsorbent bed while withdrawing a spent desorbent from the trim adsorber; discontinuing the first adsorbent bed displacement; and passing an adsorber trim fluid to the first adsorbent bed to recover the remaining treated feed therein the head; and passing a trim fluid to the trim bed to displace feed from the trim bed. An embodiment of the disclosure is one, any or all of prior embodiments in this paragraph up through the third embodiment in this paragraph further comprising combining feed displaced from the trim bed with feed that is feeding the second adsorbent bed. An embodiment of the disclosure is one, any or all of prior embodiments in this paragraph up through the third embodiment in this paragraph further comprising passing the feed stream through the second adsorbent bed to adsorb material from the feed stream onto the second adsorbent bed.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of treating a feed in an adsorption separation zone comprising at least two adsorbent beds and a trim bed, the process comprising:
displacing feed from a first adsorbent bed to a trim bed by feeding a desorbent from a second adsorbent bed to the first adsorbent bed while displacing spent desorbent from the trim bed;
discontinuing the first adsorbent bed displacement; and passing a trim displacement fluid to the trim bed to displace feed from the trim bed.

2. The process of claim 1 further comprising passing an adsorber trim fluid to the first adsorbent bed to recover the remaining treated feed; and discontinuing adsorber trim fluid to the first adsorbent bed before passing said trim displacement fluid to the trim bed.

3. The process of claim 1 wherein the adsorber trim fluid and/or the desorbent are benzene.

4. The process of claim 1 wherein the trim displacement fluid is selected from a heavy aromatics stream or a paraffinic stream.

5. The process of claim 1 further comprising:
discontinuing the displacement of feed from the trim bed with the trim displacement fluid;
passing an adsorber trim fluid to the trim bed to displace spent trim displacement fluid from the trim bed while withdrawing a spent trim displacement fluid from the trim bed.

6. The process of claim 1 further comprising combining feed displaced from the trim bed with feed that is feeding the second adsorbent bed.

7. The process of claim 1 further comprising passing an adsorber trim fluid to the first adsorbent bed to displace treated feed from the first adsorbent bed to the trim bed.

8. The process of claim 6 wherein the trim displacement fluid comprises heavy aromatics.

9. The process of claim 1 further comprising passing the feed stream through the second adsorbent bed to adsorb material from the feed stream onto the second adsorbent bed.

10. The process of claim 1 further comprising passing a desorbent stream through the first adsorbent bed and the trim bed to remove adsorbed material from the first adsorbent bed and the trim bed to regenerate the first adsorbent bed and the trim bed and withdrawing the spent desorbent stream from the trim bed.

11. The process of claim 1 wherein desorbent is pushed from the second adsorbent bed to the first adsorbent bed by feeding feed to the second adsorbent bed.

12. The process of claim 1 further comprising:
passing spent trim displacement fluid to an aromatics fractionation column to provide an overhead stream comprising benzene and a bottoms stream comprising heavy aromatics; and
recycling the heavy aromatics as a trim fluid.

13. The process of claim 1 further comprising:
passing the spent desorbent to a desorbent column to provide a bottoms stream comprising aromatics and an overhead stream comprising the desorbent.

14. A process of treating a feed stream in an adsorption separation zone comprising at least two adsorbent beds and a trim bed, the process comprising:
passing the feed stream through the first adsorbent bed to adsorb material from the feed stream onto the first adsorbent bed;
discontinuing passing feed to the first adsorbent bed;
passing the feed stream through the second adsorbent bed to adsorb material from the feed stream onto the second adsorbent bed;
displacing desorbent in the second adsorbent bed to the first adsorbent bed;
displacing feed from the first adsorbent bed to the trim bed;
withdrawing a spent desorbent from the trim adsorber;
discontinuing displacing feed from the first adsorbent bed;
passing an adsorber trim fluid to the first adsorbent bed to recover remaining feed therein; and
passing a trim displacement fluid to the trim bed to displace feed from the trim bed.

15. The process of claim 14 further comprising:
discontinuing the displacement of feed from the trim bed with the trim displacement fluid;
passing an adsorber trim fluid to the trim bed to perform a trim displacement of the trim bed while withdrawing a spent trim displacement fluid from the trim bed;
discontinuing the trim displacement of the trim bed with the adsorber trim fluid.

16. The process of claim 14 further comprising combining feed displaced from the trim bed with feed that is feeding the second adsorbent bed.

17. The process of claim 14 further comprising passing a adsorber trim fluid to the trim bed to displace the trim displacement fluid from the trim bed.

18. The process of claim 14 further comprising passing a desorbent stream through the first adsorbent bed and the trim bed to remove adsorbed material from the first adsorbent bed and the trim bed to regenerate the first adsorbent bed and the trim bed and withdrawing the spent desorbent stream from the trim bed.

19. A process of treating a feed in an adsorption separation zone comprising at least two adsorbent beds and a trim bed, the process comprising:
displacing feed from a first adsorbent bed to a trim bed by feeding feed to a second adsorbent bed to displace desorbent from the second adsorbent bed to the first adsorbent bed while withdrawing a spent desorbent from the trim adsorber;
discontinuing the first adsorbent bed displacement; and passing an adsorber trim fluid to the first adsorbent bed to recover the remaining treated feed therein the head; and
passing a trim fluid to the trim bed to displace feed from the trim bed.

20. The process of claim 19 further comprising combining feed displaced from the trim bed with feed that is feeding the second adsorbent bed.
